(19) United States
(12) Patent Application Publication
(10) Pub. No.: US 2017/0203121 A1
(43) Pub. Date: Jul. 20, 2017

(54) SINGLE-PATIENT TEXTILE DEVICE FOR PHOTOTHERAPY TREATMENT AND UNIT COMPRISING SUCH A SINGLE-PATIENT TEXTILE DEVICE

(71) Applicant: NEOMEDLIGHT, Villeurtanne (FR)

(72) Inventors: Clarisse Huyghe, Lyon (FR); Laure Peruchon, Villeurbanne (FR); Cedric Brochier, Lyon (FR); Pierre Saint Girons, Lyon (FR)

(73) Assignee: NEOMEDLIGHT, Villeurbanne (FR)

(21) Appl. No.: 15/326,101

(22) PCT Filed: Jul. 24, 2015
(86) PCT No.: PCT/FR2015/052058

§ 371 Date: Jan. 13, 2017

Related U.S. Application Data
(60) Provisional application No. 62/028,817, filed on Jul. 25, 2014.

(51) Int. Cl. A61N 5/06

(52) U.S. Cl. CPC A61N 5/0621; A61N 2005/0638; A61N 2005/063; A61N 2005/0663

(57) ABSTRACT

A single-patient textile device for providing phototherapy treatment to a patient. The device includes a back wall intended to accommodate the patient in a supine position, the back wall having an upper portion and a lower portion; a front wall attached to the lower portion of the back wall; and two lateral walls which are arranged on either side of the back wall and can be folded onto the back wall. Once folded, the front wall and lateral walls form a volume to house the patient, including an opening to enable the passing of the patient's head through the upper portion of the back wall. The front wall can be folded toward the back wall such that the front wall and the back wall each have an inner surface that can distribute the treatment light within the volume.

### CROSS-REFERENCE TO RELATED APPLICATIONS

This application is a §371 filing of PCT application PCT/FR2015/052058 filed on Jul. 24, 2015, which claims priority from U.S. Provisional Application 62/028,817 filed on Jul. 25, 2014. The disclosures of these applications are included by reference herein in their entirety.

### BACKGROUND

Field of the Invention

The invention relates to the phototherapy treatment industry.

More specifically, the invention relates to a flexible device for phototherapy treatment enabling treatment of patients, notably those with infant jaundice and Crigler-Najjar syndrome, a rare disorder.

Description of the Prior Art

Infant jaundice and Crigler-Najjar syndrome, a rare disorder, are disorders related to excessive levels of free bilirubin. Free bilirubin builds up in the body and leads to jaundice (yellow skin and eyes), which may cause neurological damage when the bilirubin level is very high. Jaundice is very often observed in newborns, although it may also be present in adults. Crigler-Najjar syndrome is a rare genetic disease of the liver. The symptomatic treatment of Crigler-Najjar syndrome consists of daily phototherapy of 10 to 12 hours, while the treatment for infant jaundice may last up to 24 hours.

In recent years, special interest has been noted in the medical world for the development and optimization of efficient phototherapy treatments for disorders caused by abnormally high levels of bilirubin. Phototherapy treatment is the most used treatment in hospitals as its implementation is far less cumbersome compared to other treatments that may be applied, such as exchange transfusion (a technique allowing for the replacement of a large portion of an individual's blood) for infant jaundice or liver transplant for Crigler-Najjar syndrome patients.

In general, phototherapy devices for the treatment of jaundice are well known to those skilled in the art. Various different devices are currently on the market. The emergence of two categories of devices may be noted, namely rigid devices which consist of a shell, and others which are more flexible such as luminous textile devices.

Flexible textile devices, because they are attractive in terms of cost and comfort, have seen the most interesting developments in recent years and have therefore managed to achieve the most significant market share gains.

In general, a so-called flexible phototherapy treatment device is composed of a visible light source that emits at wavelengths corresponding to blue light. The blue light absorbed by bilirubin enables free bilirubin to be converted into water-soluble degradation products easily excreted in urine and harmless to humans. This light source is connected by an optical connector to a cable. The cable contains a plurality of optical fibers that conduct light to the patient. The optical fibers often emerge from the cable in the form of a “pad” which is intended to be inserted into a housing. These devices, which are flexible, have seen recent developments aimed to improve and optimize phototherapy treatment efficiency.

Notably known are devices such as those described in WO 2004/064924. These devices comprise a so-called padded area in their lower portion which provides support to the patient. In fact, during the treatment the patient moves. In practice, it has been noted that infants tend to slide toward the lower portion of the device. These devices specifically make it possible to prevent the patient from sliding within the structure. The risk of exposure of sensitive parts of the patient's body, such as their head and more particularly their eyes, is therefore significantly diminished.

However these devices, which comprise a so-called padded area, are designed to accommodate the patient preferably in a fetal position. Nevertheless, a fetal position implies that certain areas of the patient's body are not exposed to the light emitted by the light source. Consequently, these devices do not allow for uniform exposure over the whole patient's body. Treatment effectiveness is not therefore maximized by these devices.

Also known from this document are flexible devices, such as blankets that may be wrapped around the patient, and which can accommodate an optical fiber-based pad with a view to phototherapy treatment. Nevertheless, this type of device does not allow for the achievement of a high intensity treatment over long periods of time, for example 10 to 12 hours. Specifically, the proposed structure has no means to remove the heat generated by exposure to light.

Also known in the prior art are other flexible devices such as the one described in WO 2013/068518, comprising a layer made of phase-change materials, which enables treatments to be carried out over longer time periods and also with higher light intensity. Although it improves the patient's comfort from a purely thermal point of view thanks to automatic temperature control, this type of device does not allow the patient to be held in an optimal position. Given the volume of illumination offered by this type of device, lateral movements do not reduce treatment efficiency. However, the patient's sliding movements toward the lower portion of the device may not be excluded and constitute an important drawback. In fact, it is essential to prevent the patient's head, more precisely their eyes, from being exposed to light rays. However, these devices fail to avoid this scenario. Accordingly, this type of device is limited as it fails to prevent risky situations of exposure of the patient's sensitive parts due to the fact that it does not restrict the patient's movements during treatment.

Currently, this flexible phototherapy device for the treatment of jaundice fails to restrict the patient's movements only within the volume of illumination in at least two directions, while preventing the patient's containment within the lower portion of said device, for the purpose of optimizing the patient's area of exposure.

Accordingly, the need for a flexible and comfortable phototherapy treatment device, which allows for efficient patient exposure over a longer period, can be easily understood.

### DISCLOSURE OF THE INVENTION

The object of the invention is therefore to provide a flexible device that allows for obtaining such a result.

To this end, the invention relates to a single-patient textile device for providing phototherapy treatment to a patient, said device comprising:
a back wall intended to accommodate the patient in a supine position; said back wall comprising an upper portion and a lower portion;
a front wall attached to the lower portion of the back wall;
two lateral walls which are arranged on either side of the back wall and can be folded onto said wall;
   once folded, the front wall and the lateral walls form a volume to house the patient, including an opening to enable the passing of the patient's head through the upper portion of the back wall.

According to the invention, the single-patient textile device is characterized in that said front wall can be folded onto said back wall, such that the front wall and the back wall each has an inner surface that can distribute the light within said volume.

In other words, thanks to this specific structure, the single-patient textile device allows for efficient positioning of the patient, in a supine position, on the back wall. This single-patient textile device has a length that is sufficient to accommodate infants of different sizes. Once the patient has been properly positioned, the practitioner can thus fold the front portion in order to cover the whole of the patient's body.

Whole of the body means the entire body except for the head, which emerges from the opening in the upper portion of the single-patient textile device. The patient finds themselves lying, “sandwiched” between the back portion and the front portion. The single-patient textile device is therefore designed to position the patient in a supine position in order to direct the emitted light over the whole of their body. Thanks to the presence of an inner layer, which allows for the transmission of the light on both the back and the front walls, the patient is entirely exposed. Advantageously, the inner surface disseminates at least 75% of the received light.

The folded lateral walls define the volume within which the patient is housed. Advantageously in practice, the length of the lateral walls is less than the length of the back wall, such that the volume includes openings into the lower portion of the back wall. These openings notably allow for the removal of the heat generated by the light during the treatment and more specifically during a long exposure. The single-patient textile device permits some air circulation which allows for better patient comfort during treatment without greatly increasing the risks of light leakage.

In one embodiment, the single-patient textile device comprises means for holding the patient in place which are integral to the back portion.

Advantageously in practice, said holding means are composed of at least three straps sewn onto the back portion. Two side straps allow the patient's pelvis to be held onto the back portion, preferably in the center thereof. A third strap allows for holding the patient's crotch and is intended to be attached to the two transverse straps by an adhesive system or similar.

During treatment, the patient is thus restricted in terms of movements in at least two directions, namely the transverse direction and the longitudinal direction. This prevents the patient from any sliding toward the lower portion, and thereby the exposure of the patient's sensitive parts such as the face, and more specifically the eyes.

According to a preferred embodiment, the straps protect the patient's genitals and to this effect they are advantageously made based on a nonwoven material such as a “spunbond” or any other similar material fulfilling the same function.

In one specific embodiment, the position-holding means may form a layer. In fact, treatments are usually carried out over long periods and the presence of a layer integral to the device is therefore relevant.

In practice, the back wall and the front wall are attached by a folding area comprising at least one transverse fold, which allows the front portion to be folded onto the patient's body area to be treated. The folding area facilitates the folding movement of the two walls and is preferably located above the patient's feet.

The back wall and the front wall comprise at least two superimposed layers; namely a layer that forms the inner surface and a second layer that forms the outer surface of the single-patient textile device.

Advantageously, the inner surface of the single-patient textile device is made primarily of a nonwoven textile that advantageously is soft to the touch, which gives comfort properties to the textile device.

At the same time, the outer surface may be made of a textile or a nonwoven material that reduces light leaks to maximize phototherapy treatment efficiency and limit the risks related to the exposure of eyes to intense light.

In practice, the back wall and the front wall each has a housing that can accommodate a light emitting source, respectively. Thus, as the patient is in direct contact with said two walls, the emitted light is then primarily directed toward their body. This positioning of the light-emitting sources allows the system to restrict the light emission toward the outside of the volume defined by the single-patient textile device.

Furthermore, the device eliminates the risks related to the exposure of sensitive parts of the patient's body, since the genitals as well as the eyes are not exposed to light during treatment.

In practice, the upper portion of the back wall comprises two braces enabling said back wall to be secured to the front wall when folded. Thus, once said braces have been folded, the single-patient textile device has a limited and controlled upward opening which prevents the light emitted within the volume from traveling toward the sensitive areas of the patient's body and more specifically toward the eyes.

In practice the front wall, the lateral walls and the braces each has fastening means to secure them to one another.

The fastening means are preferably of the soft Velcro type (such as those used for newborn diapers). They notably allow the single-patient textile device to be held efficiently in a “folded” position during treatment, avoiding any openings despite potential patient movements. In addition they allow, in an open position, the unfolding of the lateral walls to improve air circulation.

In a variant, the single-patient textile device comprises a device for holding the sources that distribute light within housings. Advantageously, the holding device consists of two integral straps that, when interlaced, allow the luminous members to be held within the housings of the single-patient textile device. The proper positioning of these light sources is thus ensured irrespective of the patient's movements.

In practice, a unit for providing phototherapy treatment to a patient comprises at least:
one single-patient textile device such as that defined above;
one light source;
at least one luminous member made up of optical fibers; the optical fibers being connected to the light source by an optical fiber cable equipped with an optical connector and at least one luminous member being suitable to be integrated into at least one of the housings of the single-patient textile device.

Advantageously, the optical fibers emit light at a wavelength of between 400 nm and 520 nm, preferably between 430 and 490 nm (wavelength corresponding to the maximum absorption of bilirubin).

### BRIEF DESCRIPTION OF FIGURES

The invention will be better understood upon reading the following description, given solely as a non-limiting example, and with reference to the accompanying figures in which:

FIG. 1 is a schematic top view of a single-patient textile device for providing phototherapy treatment, unfolded, in a preferred embodiment of the invention;

FIG. 2 is a schematic bottom view of the single-patient textile device in FIG. 1;

FIG. 3 is a schematic view of the single-patient textile device in FIG. 1, showing the case where said luminous members are inserted into said housings and said front wall is folded;

FIG. 4 is a schematic top view of the device in FIG. 1, showing the case where said holding means is folded;

FIG. 5 is a schematic top view of the single-patient textile device in FIG. 1, folded, within which a patient is in a supine position;

FIG. 6 is a schematic view of a phototherapy treatment unit integrating said single-patient textile device in FIG. 1.

### METHOD FOR IMPLEMENTING THE INVENTION

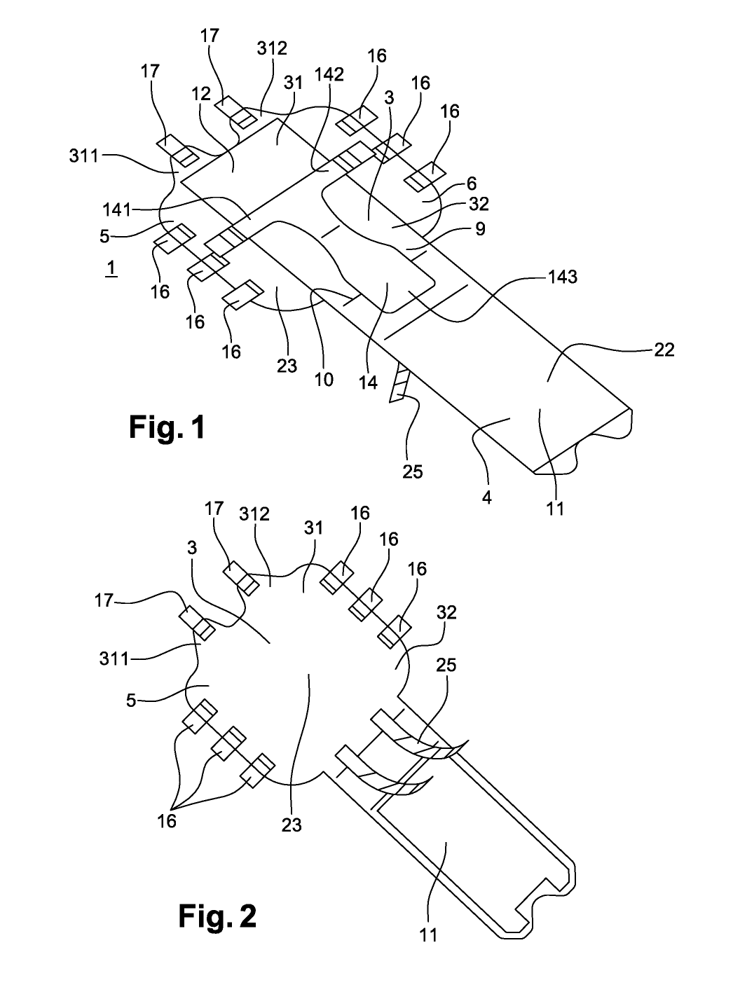

FIG. 1 shows a single-patient textile device 1 for providing phototherapy treatment which comprises a back wall 3 with an upper portion 31 and a lower portion 32.

The single-patient textile device 1 also has a so-called front portion 4 which is attached to the back portion 3 through its lower portion 32.

The front wall 4 is attached to the back wall 3 through a so-called folding area 9 with a transverse fold 10. Advantageously in practice, the front wall 4 can be folded onto the back wall 3.

The back portion 3 and the front portion 4 are composed of two layers 22, 23. Layer 22 forms the inner surface of the single-patient textile device 1, while the layer 23 forms the outer surface of the single-patient textile device 1.

The layer 22 is advantageously made of a viscose and/or cotton-based nonwoven textile. For example, the layer 22 is made of a material such as “spunlace” or similar.

The layer 22 can let the light pass therethrough during phototherapy treatment. The desired result is to minimize as much as possible the absorption or reflection of light by the layer 22. To this end, the layer 22 advantageously has a maximum surface mass density of 60 g/m². In this case, the layer 22 is sufficiently thin to allow at least 75% of the light to pass therethrough, and thereby increase the efficiency of the phototherapy treatment for the patient 2.

During long term treatment (up to 24 hours continuously) at high light intensity, the patient is in an environment where the temperature conditions can be relatively high causing the patient to sweat. The single-patient textile device 1 therefore has water absorption properties sufficient to ensure patient comfort. The layer 22 has water absorption greater than 400%, advantageously greater than 800%.

As shown in FIG. 2, the layer 23 that forms the outer surface of the back wall 3 and front wall 4 is made of a mainly polypropylene-based nonwoven textile. The material forming the layer 23 is advantageously “spunbond” or other similar material.

Since the object is to focus a maximum of light toward the patient during treatment, the layer 23 also has opacity properties that allow for achieving such a result.

Furthermore the layer 23, which is exposed to the external environment, is not directly in contact with the patient. It has water absorption capacity that is less than that of the layer 22, advantageously between 100 and 150%.

The back wall 3 is intended to accommodate the patient during treatment. In order to remedy a major treatment risk, namely that of exposing sensitive parts of the patient's body such as the eyes, the single-patient textile device 1 advantageously comprises a holding means 14 for the patient's position which is integral to the back portion 3.

As shown in FIG. 1, this holding means 14 comprises three straps 141, 142, 143.

As shown in FIG. 4, two side straps 142, 143 hold the patient's pelvis, thus restricting lateral movement within the single-patient textile device 1. A third strap 141, which can be folded, allows for maintaining the patient's crotch, thus preventing the patient from any sliding toward the lower portion of the single-patient textile device 1.

Advantageously in practice, the three straps 141, 142, 143 are sewn to the back portion 3 and are made of a material identical to that of the outer surface 23.

In a specific embodiment, the holding means forms a layer for newborns composed of the three straps.

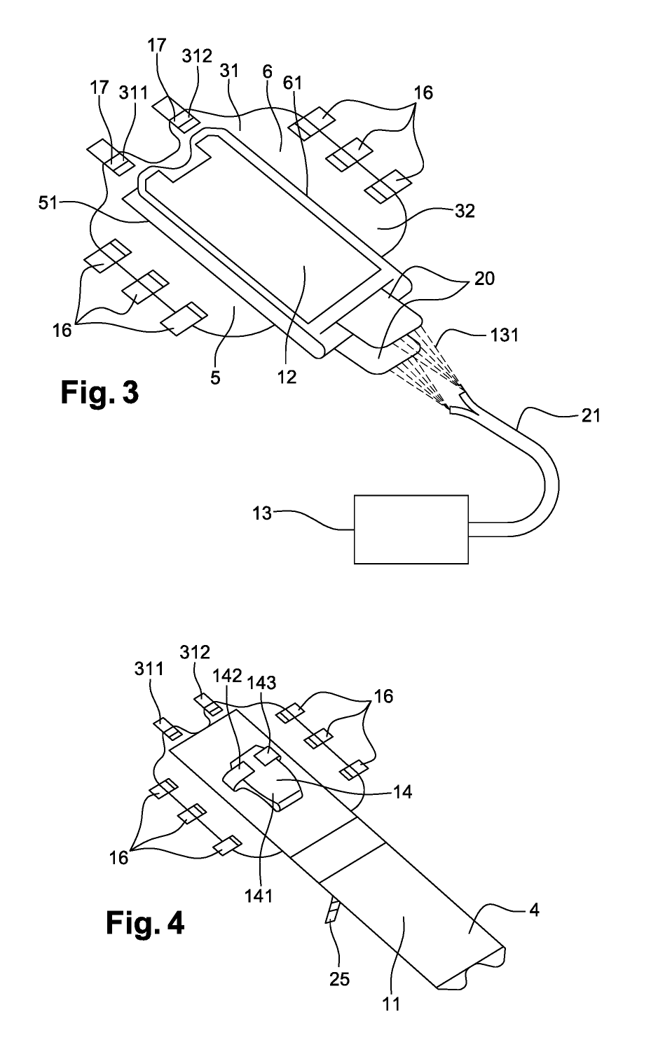

As shown in FIG. 3, the back wall 3 and the front wall 4 each has a housing 11, 12 respectively, which can accommodate the luminous members 20 of the optical fibers 131 disseminating the light emitted by the light source 13. Advantageously in practice, said housings 11, 12 are in the form of woven pockets of a complementary shape to that of the luminous members 20.

Side walls 5, 6 are also arranged on either side of the back wall 3. They are made of an identical material to that of the outer layer 23, namely an opaque mainly polypropylene-based woven textile. These lateral walls thus have physical, mechanical and chemical properties similar to the outer layer 23.

Furthermore, the lateral walls 5, 6 can fold onto the back wall 3. Therefore they each have a folding area 51 and 61, preferably longitudinal relative to the back wall 3.

The single-patient textile device 1 also has two braces 311, 312 to secure the upper portion 31 of the back wall 3 to the front wall 4. Said braces 311, 312 are preferably made of a material similar to that of the outer layer 23.

As shown in FIG. 1, the lateral walls 5, 6, the braces 311, 312 and the front wall 4 are equipped with fastening means 16, 17, advantageously of the soft Velcro type or similar.

Thus once the walls have been folded, the volume 7 is then formed within the single-patient textile device 1 and defines the volume 7 for the patient housing.

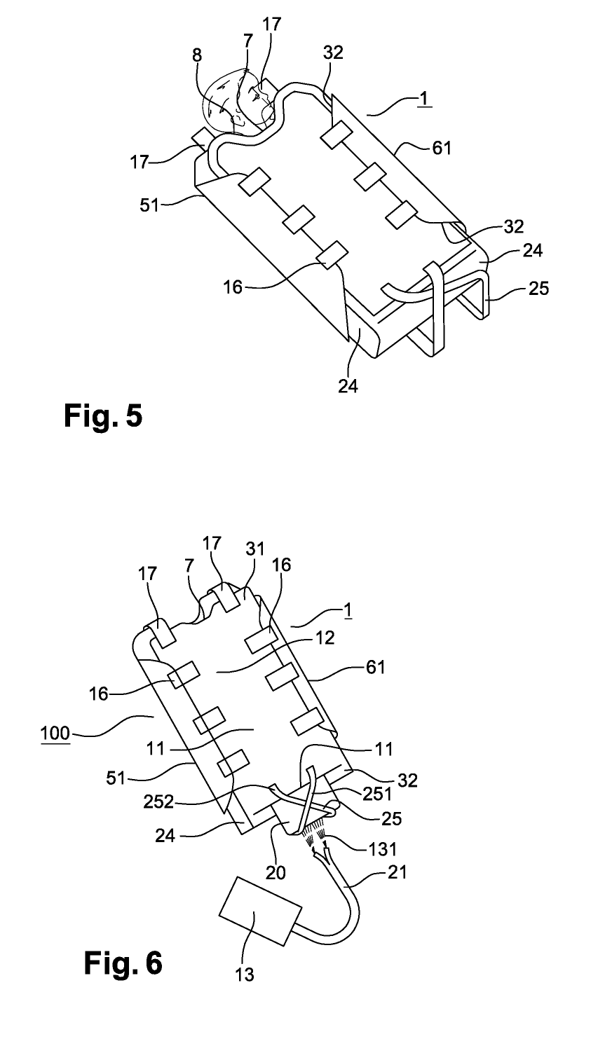

As shown in FIG. 5, the patient's head 2 emerges from the single-patient textile device 1 through an opening 8.

The length of the lateral walls 5, 6 is less than the length of the back wall 3, such that the volume 7 includes openings 24 into the lower portion 32 of the back wall 3.

Advantageously in practice, the length of the lateral walls 5, 6 is between 350 and 400 mm, preferably 360 mm.

Furthermore, the length of the back wall 3 is between 400 and 500 mm, preferably 460 mm.

Each of the openings 24 removes heat such that the temperature within the single-patient textile device 1 can be adjusted by the caregiver: in the folded position, the calories are conserved within the device 1, and the side openings 24 serve to lower the temperature if necessary.

The single-patient textile device 1 has, in addition, a fastening device 25 of the luminous members 20 within the housings 11, 12, which consists of two straps 251, 252 intended to tighten the lower portion 32 of the device 1. The straps 251, 252 are preferably made of spunbond.

A specific embodiment of the invention has been disclosed. However, many versions are possible, such as those below, taken alone or in combination.

A single-patient textile device has been disclosed, with lateral walls on either side of the back portion. Alternatively, the sidewalls may be provided on either side of the front portion intended to be folded onto the back portion.

A system of straps has been disclosed, as a means for holding the patient in place. Nevertheless, any other type of holding system may be integrated into the single-patient textile device according to the invention.

A folding area has been disclosed, comprising a single transverse fold. In another embodiment, the folding area has at least two transverse folds.

Soft Velcro type fastening means (hoop & loop system) have been disclosed. However, any other types of fastening means known in the prior art may be integrated into the single-patient textile device of the invention.

As shown in FIG. 6, a device 100 for providing phototherapy treatment to a patient 2 comprises at least:
one single-patient textile device 1 as previously disclosed;
one light source 13;
at least one luminous member 20 made up of optical fibers 131; said optical fibers 131 being connected to the light source 13 by an optical fiber cable 21 equipped with an optical connector and said at least one luminous member 20 being suitable to be integrated into at least one of the housings 11, 12 of said single-patient textile device 1.

The light source 13 has a spectral irradiance that may vary from 30 μW/cm²·nm to 100 μW/cm²·nm at a wavelength between 400 and 520 nm.

The peak of the light wavelength emitted by the source 13 is between 400 nm and 520 nm which corresponds to blue color within the visible light spectrum and to the bilirubin absorption peak.

The optical fibers 131 are preferably made of a polymer material in order to conduct the light emitted by the light source 13 effectively to the luminous members 20.

For example, the optical fibers have a core coated at least partially by a sheath and can be made of polymethylmethacrylate (core) and a fluoropolymer (sheath).

The optical fibers 131 may be subjected to a side treatment along their length which is tailored to the application. Advantageously in practice, the fibers 131 emit unidirectionally toward the patient 2.

In addition, the fibers 131 are advantageously arranged in parallel within the luminous members 20.

The cable 21 is preferably made of an opaque polyolefin-based material, covered with a white polyurethane film.

Advantageously in practice, the luminous members 20 have a rectangular shape and have a length between 300 and 600 mm and a width between 200 and 500 mm. The dimensions of the luminous members 20 are slightly smaller than the dimensions of the housings 11, 12 that accommodate them.

Its composition and simplicity of design cause the single-patient textile device 1 to be advantageously intended for single use.

Nevertheless, the textile device may also undergo several washings in order to be reused.

1. A single-patient textile device for providing phototherapy treatment to a patient, the device comprising:
a back wall intended to accommodate the patient in a supine position, the back wall comprising an upper portion and a lower portion;
a front wall attached to the lower portion of the back wall; and
two lateral walls arranged on either side of the back wall and foldable toward the back wall;
wherein, once folded, the front wall and two lateral walls form a volume with the back wall to house the patient, the volume having an opening to enable the passing of the patient's head through the upper portion of the back wall (3); and wherein the front wall and the back wall each comprise an inner surface adapted to distribute light from a light source to within the volume.

2. The device according to claim 1, wherein a length of the lateral walls is less than a length of the back wall, such that the volume includes openings.

3. The device according to claim 1, wherein the device further comprises a means to hold the patient in place, the means mounted to the back wall.

4. The device according to claim 3, wherein the position holding means comprises straps.

5. The device according to claim 1, wherein the back wall and the front wall each comprise at least two superimposed layers, wherein a first layer of the at least two superimposed layers forms the inner surface of each of the front wall and the back wall and a second layer of the at least two superimposed layers forms an outer surface of the single-patient textile device.

6. The device according to claim 1, wherein the inner surfaces of the front wall and the back wall disseminate at least 75% of the light from the light source.

7. The device according to claim 1, wherein the light source comprises a plurality of light sources, and wherein the back wall and the front wall each comprise a housing having one of the plurality of light sources.

8. The device according to claim 1, wherein the upper portion of the back wall comprises two braces to secure the upper portion of the back wall to the front wall.

9. The device according to claim 8, wherein the device further comprises fastening means.

10. The device according to claim 7, wherein the device further comprises devices for holding one of the plurality of light sources within each of the housings.

11. A unit for providing phototherapy treatment to a patient, the unit comprising:
the single-patient textile device according to claim 1;
a light source; and
a luminous member having optical fibers, the optical fibers being connected to the light source by an optical fiber cable equipped with an optical connector, and wherein the luminous member is adapted to be integrated into the single-patient textile device.

12. The unit according to claim 11, wherein the light source emits a wavelength between 400 nm and 520 nm.
